(19) United States
(12) Patent Application Publication
(10) Pub. No.: US 2017/0246076 A1
(43) Pub. Date: Aug. 31, 2017

(54) APPLIANCE FOR TREATMENT OF FACIAL SKIN CONDITIONS

(71) Applicant: L'Oreal, Paris (FR)

(72) Inventors: Zane Miller, Seattle, WA (US); Matthew O. Winterscheid, Seattle, WA (US)

(21) Appl. No.: 15/054,992

(22) Filed: Feb. 26, 2016

(51) Int. Cl. A61H 9/00; A61N 5/06; A61N 1/32; A61H 23/02

(52) U.S. Cl. CPC A61H 9/0057; A61H 23/02; A61N 5/0616; A61N 1/328; A61N 1/325; A61H 2201/10; A61H 2205/022; A61H 2201/1207; A61H 2201/5097; A61N 2005/0647; A61N 2005/0651

(57) ABSTRACT

A mask member which fits over a portion of the face of a user. A retaining member holds the mask in place on the user's head. Eye portions allow the user to see while wearing the mask. A treatment mechanism is positioned on an interior surface of the mask for skin treatment including treatment around the eyes. A control mechanism controls the treatment mechanism.

### TECHNICAL FIELD

This invention relates generally to a appliances for facial skin treatment, including skin treatment around the eyes, and more particularly to such appliances which are hands-free and permit the user to be mobile for performing various tasks.

### BACKGROUND OF THE INVENTION

Historically, human skin ages over time, particularly the facial skin, including around the eyes. Certain glasses, such as sunglasses, are used to protect the skin surrounding the eye area as well as the eyes. Treating these areas of skin with formulations helps slow down the breakdown of collagen fibers in the skin area. While sunglasses shield the skin from UV radiation, they do not treat the skin.

There are various known treatments for preventing or slowing the breakdown of collagen in facial skin, including LED light and massage therapy. LED light technology is a modern treatment which uses light to penetrate into the epidermis of the skin, particularly at specific wave lengths. Collagen treatment can also be effective with pressure (massage) stimulation. Massage therapy helps to increase blood flow by the use of changes in physical pressure being applied to skin and muscle tissue.

However, current known treatments for facial skin, around the eyes in particular, often require the use of the user's hands for application of treatment formulations or for hand held devices, such as brushes. Other treatment devices do not require the use of the user's hands, such as LED light panels, but these require the user to be positioned in front of a panel for a selected period of time. Such devices are not designed to be mobile and require the user to sit for a period of time without significant movement. These are significant disadvantages in current facial skin treatment. Hence, it would be desirable for a skin treatment appliance to not require the use of hands, and permit the user mobility during treatment.

### SUMMARY OF THE INVENTION

Accordingly, the present mask assembly for treatment of facial skin conditions comprises a mask member which fits over a portion of the face of a user including skin surface around eyes of the user; a retaining member for holding the mask member in place on a user's head; eye portions allowing the user to see while wearing the mask member; at least one treatment mechanism positioned on an interior surface of the mask for skin treatment, including around the user's eyes; and a mechanism for controlling the treatment mechanism.

### BRIEF DESCRIPTION OF THE DRAWINGS

FIG. 1 is a generalized schematic view of the facial skin treatment appliance of the present invention.

FIG. 2 is a schematic view of the facial skin treatment appliance using electrostimulation treatment.

FIG. 3 is a schematic view of the facial skin treatment appliance using magnet treatment elements.

FIG. 4 is a schematic view of the facial skin treatment appliance using massage therapy arrays.

FIG. 5A is a side elevation view of one arrangement of the actuator portion of the massage embodiment of FIG. 4.

FIG. 5B is a side elevation view of another arrangement of the actuator portion of the massage embodiment of FIG. 4.

FIG. 6A is a side elevational view of an oscillating massage array.

FIG. 6B is a front view of an oscillating massage array.

FIG. 6C is a schematic view of the oscillating massage array.

FIG. 7A is a simple schematic diagram one LED treatment array.

FIG. 7B is a simple schematic diagram showing a multiple zone LED treatment array.

FIG. 7C is a simple schematic diagram showing a system for individual LED control.

FIG. 8 is a block diagram showing control of the appliance by a blue-tooth or wifi enabled smart phone or remote control device.

FIG. 9 is a block diagram showing the sequence of steps in use of the appliance.

FIG. 10 is a simplified schematic diagram showing illustrating remote control of the appliance with a blue-tooth or wifi module.

FIG. 11 is a simple schematic diagram showing a system for updating the treatment protocols for the appliance.

FIG. 12 is a diagram showing a control display of an appliance with a remote device.

FIG. 13 is a schematic diagram illustrating a system of data collection using the treatment appliance.

FIG. 14 is a schematic diagram illustrating use/storage of collection data of FIG. 13.

### BEST MODE FOR CARRYING OUT THE INVENTION

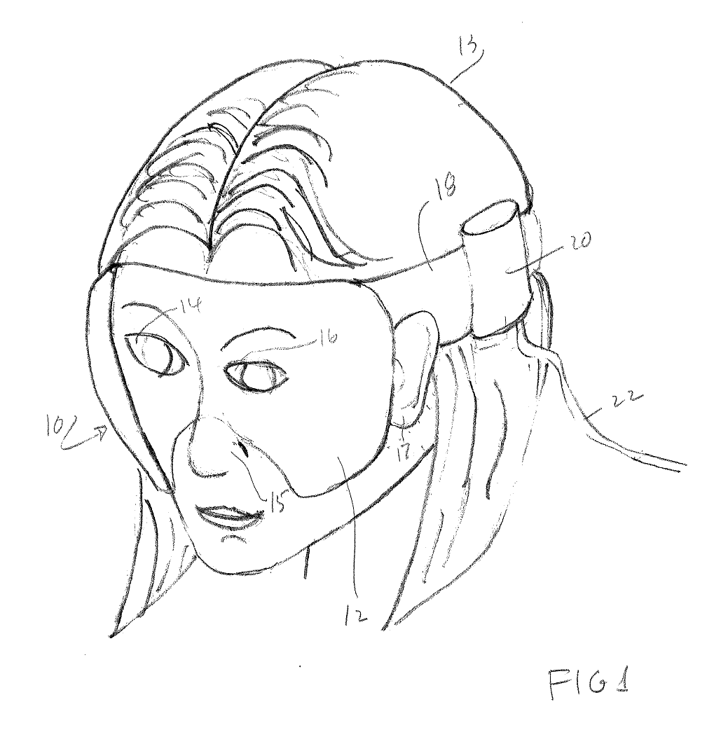

FIG. 1 shows in general the appliance of the present invention at 10. It includes a mask portion 12 which covers the upper facial portion of a user's head 13, with openings 14, 16 for the eyes and a stretchable headband 18 which releasably secures the mask to the head of the user. The masks fits around the nose 15 and typically extends to the ears 17 of the user. Power is provided to the mask by either a battery pack 20 or from an external source which is connected to the mask via a cord 22. The basic concept is a mask type of appliance or assembly which is capable of providing treatment for the facial skin, particularly around the eyes, without the need for hands and while permitting the user mobility. The mask 12 can be a conventional mask configured to fit most users or the user's face could be scanned in 3D and a custom mask could be printed and used. Waveguides or light pipes could also be printed into the structure of the mask.

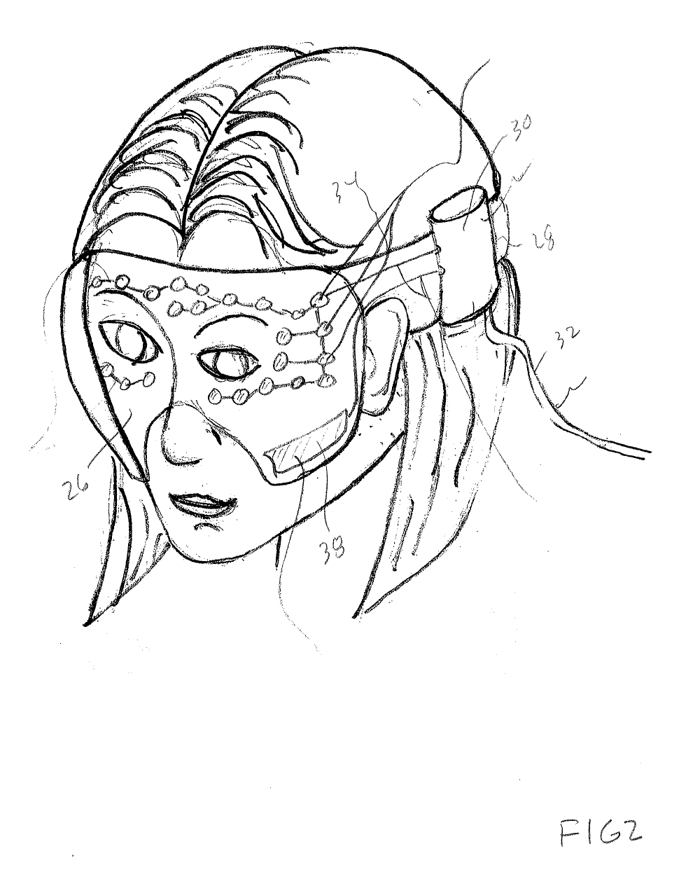

FIG. 2 shows a first specific treatment embodiment, which includes a conventional mask member 26 which comprises various flexible materials which can be fitted to a user's face or a custom mask as indicated above. The mask may or may not include a sealing member around the periphery thereof, sealing the mask to the face. Mask 26 is maintained on the user's head by a stretchable cord or other attachment member 28. Power is provide to the unit by battery pack/control unit 30 or provided through a power cord 32 from a remote source such as a wall plug. A plurality of source electrodes 34-34 are positioned on the interior portion of the mask with electrical lines 36-36 connecting the plurality of electrodes. The electrodes can be located in various positions, but would typically be at least around the eyes of the user. A return electrode is shown at 38. The control unit 30 may include an on/off switch which controls the application of micro currents with current densities at or below 0.5 mA/cm² to the facial skin area. This is for treatment by what is known as electro-stimulation or iontophoresis.

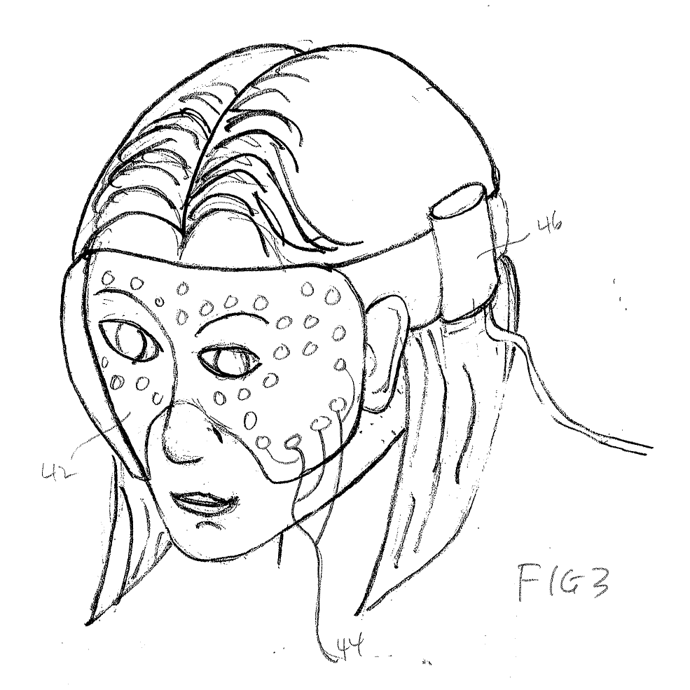

FIG. 3 shows another treatment embodiment using magnets. In this embodiment, a conventional mask 42 includes a plurality of magnets 44-44 on the inner surface of the mask. The magnetic action provides treatment for the facial skin, including the skin in the immediate vicinity of the eyes. The magnets 44 can be controlled, on-off, to produce an intermittent magnetic action by a power/control unit 46.

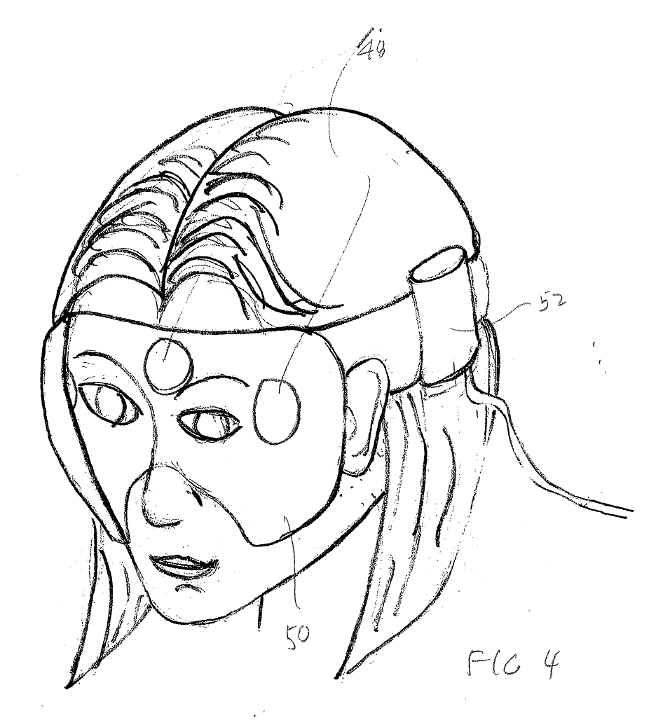

FIG. 4 shows another embodiment which includes massage therapy arrays 48-48 provided on the inner surface of a mask 50, controlled by a power/control unit 52.

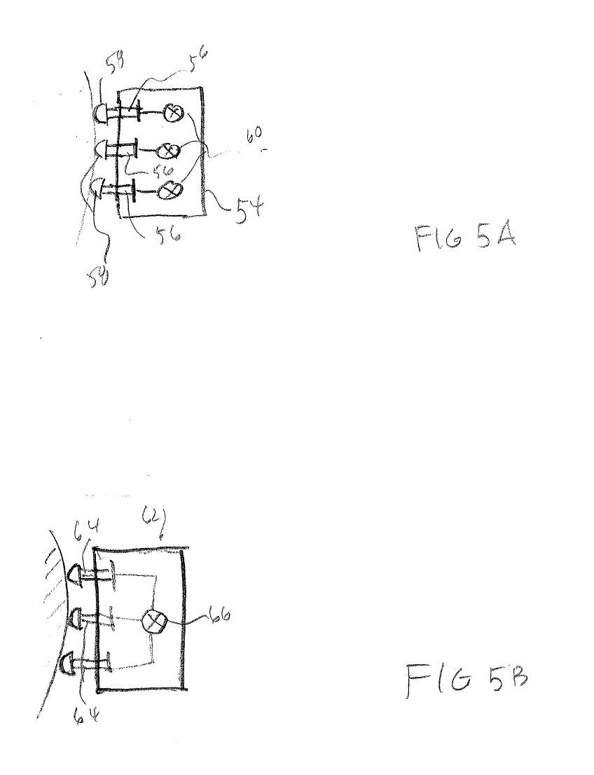

FIG. 5A shows a massage unit 54 with a plurality of actuators 56-56 and associated massage tip units 58-58 which contact the skin. The massage actuators can be either solenoid or pneumatic and are controlled by individual actuator switches 60.

FIG. 5B shows a massage unit 62, including a plurality of massage tips 64-64 controlled by a single control element 66.

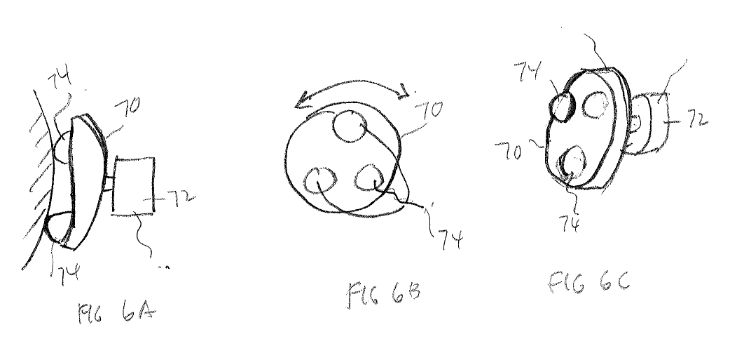

FIGS. 6A-6C show several views of an oscillating massage array 70 controlled by a motor 72 with individual massage contacts 74-74. There are three contacts in this unit, although this number can be varied, the oscillating action is through a selected angle within the range of 1-16° peak-to-peak.

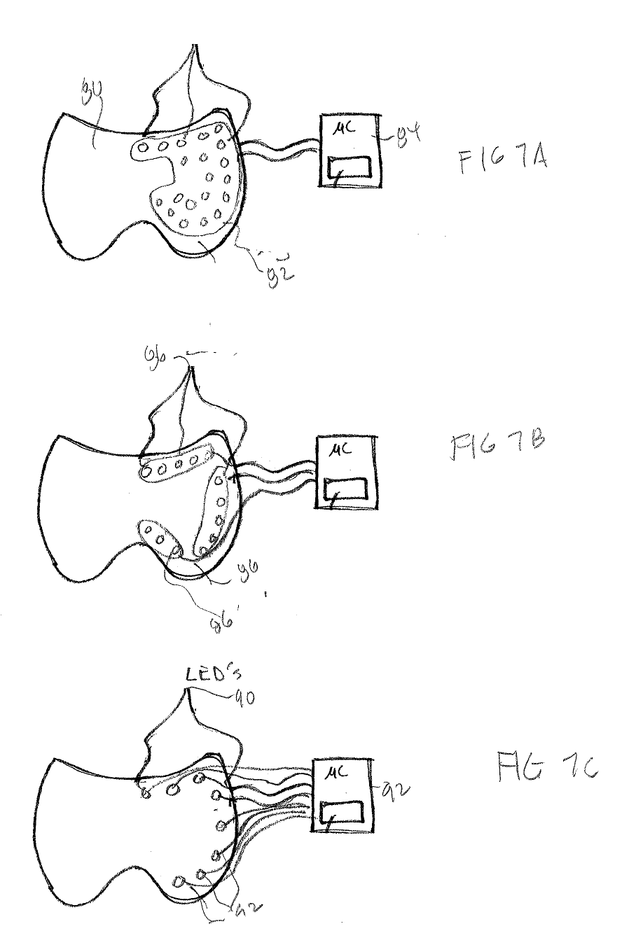

FIGS. 7A-7C show embodiments which use a plurality of LEDs for light therapy treatment embedded on the inner surface of a mask 80.

FIG. 7A shows an embodiment with a single zone 82 of LEDs, controlled by microcontroller 84. The zone can include the entire mask or a portion thereof. 7A shows a half-mask zone.

FIG. 7B shows an LED embodiment having multiple zone arrangements 86-86 with each zone including a plurality of individual LEDs, the zones positioned at desired locations in the mask, with a microcontroller 88.

FIG. 7C shows an embodiment with a plurality of LEDs 90 with each individual LED being separately controlled by microcontroller 92.

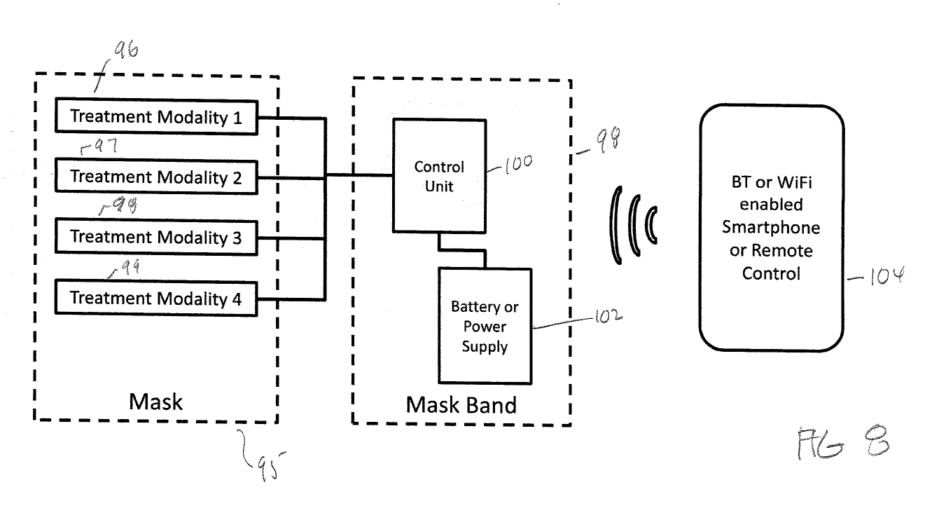

FIG. 8 shows another arrangement, shown representationally, where the mask 95 includes a plurality of embodiments, referred to as modalities 96,97, 98, and 99. While the arrangement shows four modalities, such as those discussed above, fewer or a greater number of modalities could be included. Attached to the mask band 98 is a control unit 100, with a battery or other power supply 102. The arrangement of FIG. 8 may be controlled with a Bluetooth or wifi enabled smart phone or a remote control 104. With the arrangement, the user can change modalities by use of a smart phone, without any wire connections, making it very convenient for use.

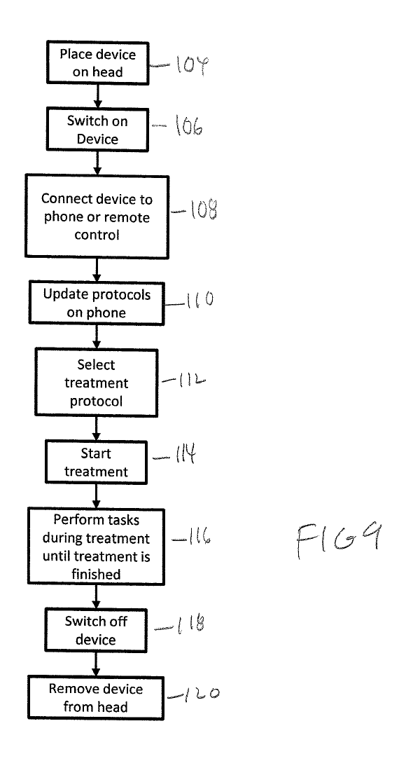

FIG. 9 shows the sequence of steps for typical use of the appliance. First, the appliance/device is placed on the user's head at 104. The user switches on the device at 106 and then connects it to a phone or remote control device, if desired, at 108. Updated protocols for one or more of the treatments, if desired, is then obtained from a remote site, at 110. One treatment protocol is selected by a user, as shown at 112. The treatment is then begun at 114. Tasks can be performed hands free and mobile by the user during treatment, at 116. This will continue until the treatment is finished. At that point the appliance is switched off at 118 and the appliance removed from the user's head, at 120.

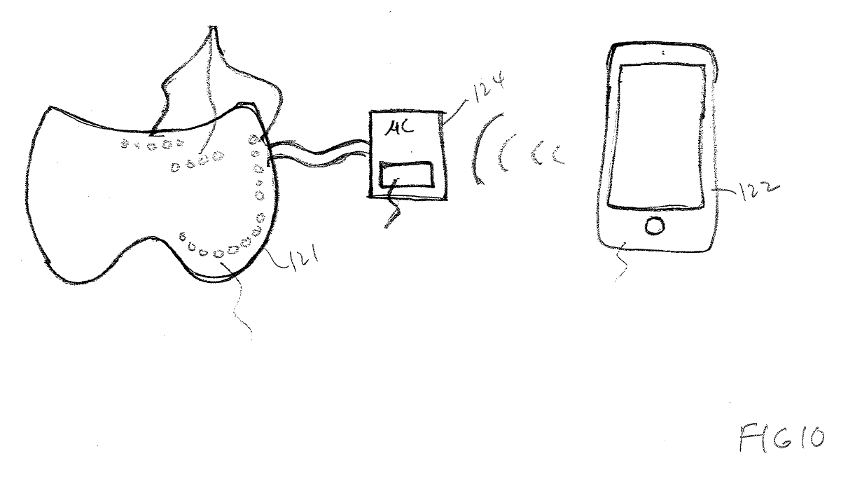

FIG. 10 shows control of the mask appliance 121 remotely by a blue-tooth phone. A wifi enabled device 122 communicates with a bluetooth or wifi module 124 on the appliance.

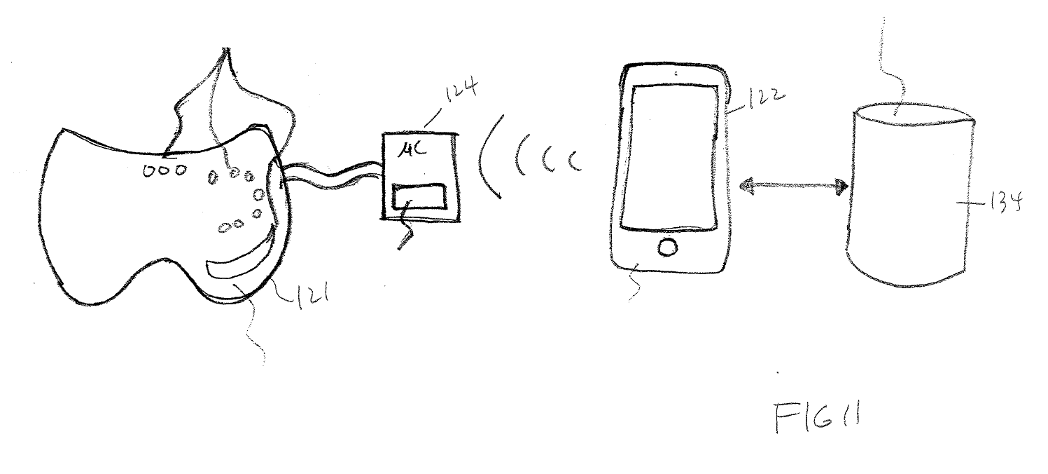

FIG. 11 shows a system similar to that of FIG. 10, but includes the capability of updating the protocol treatment arrays from a remote database 134. In such arrangement, the specific treatment regimen can be updated with new protocols as they are developed. This can be done with any treatment modality. As an example, the timing (on/off) can be controlled or the particular LED sequence can be controlled. The same kinds of updated protocols can be provided for the other treatment arrays.

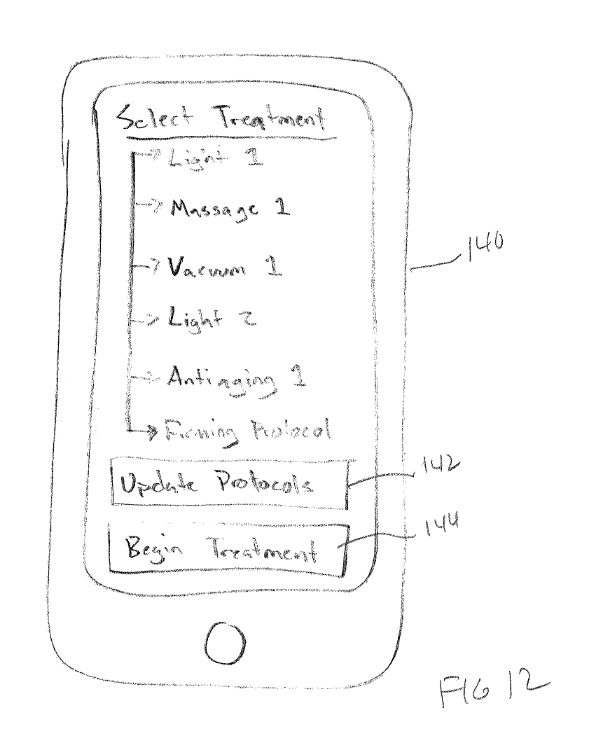

FIG. 12 shows the display of a smart phone, smart appliance or similar device 140 for use as part of the system, listing the various treatments. The user is able to choose/select a particular treatment of his/her choice among those available with a multiple treatment mask. There is an update protocol command 142, and a begin treatment switch 144 on the display.

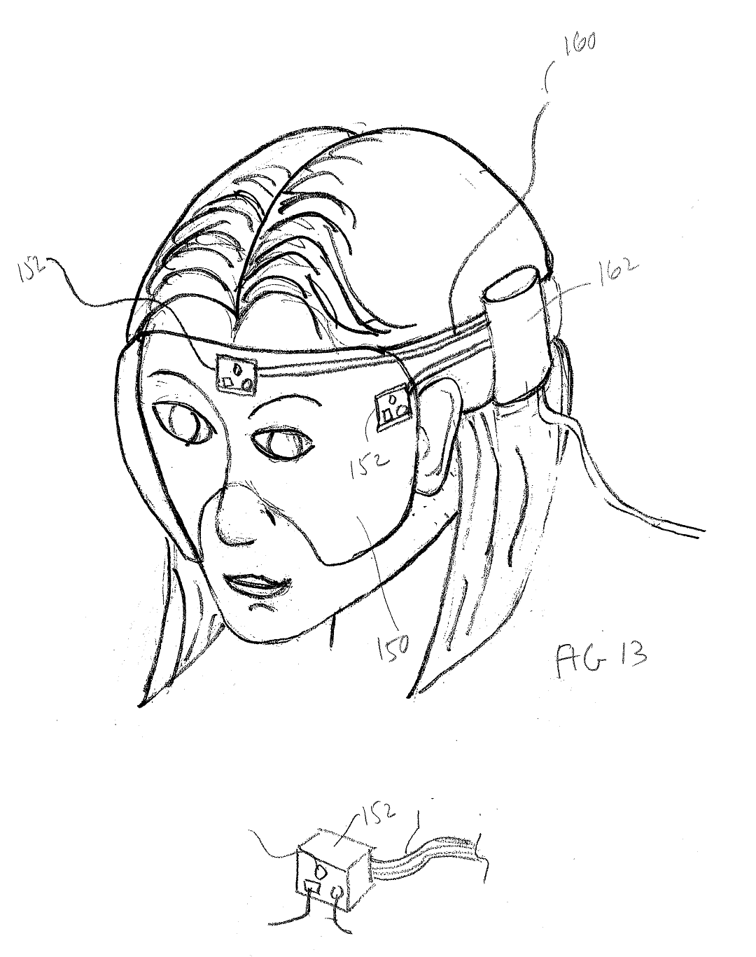

FIG. 13 shows a treatment appliance with a data collection capability. The mask 150 includes data collection units 152. These could include a camera for skin condition, including wrinkles, skin color; also sensors and LEDs for various skin conditions such as elasticity, skin hydration and skin oil condition, among others. The device includes communication lines 160 to a control unit 162.

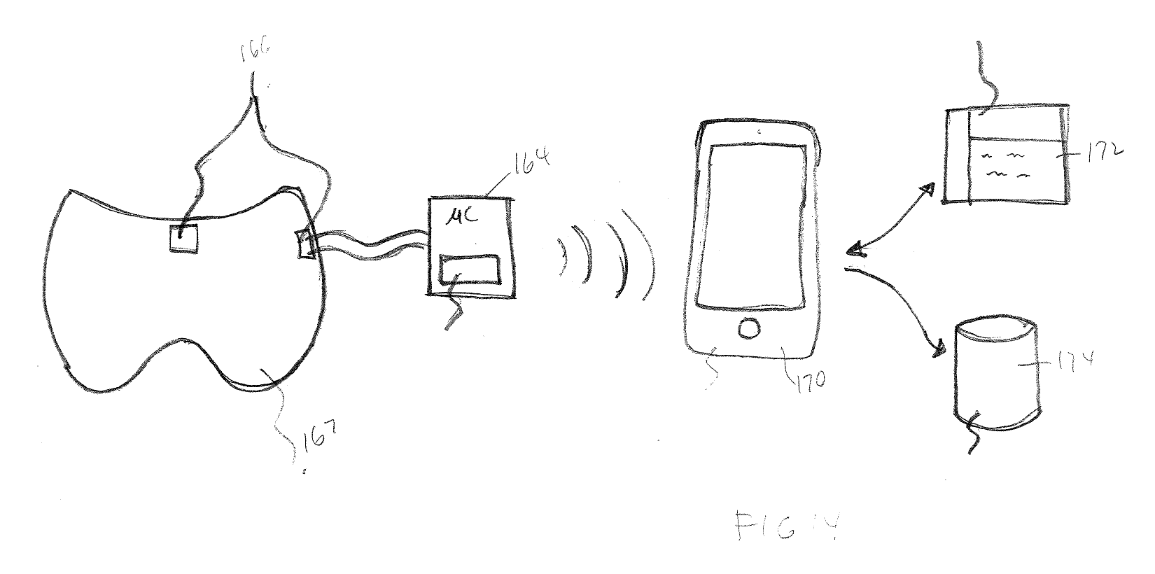

FIG. 14 shows additional elements in the data collection system. The system could include a microcontroller 164 bluetooth or wifi module for the various sensor and camera units 166 which are positioned on the interior surface of the mask 167. The microcontroller 164 communicates with a Bluetooth or wifi enabled smart device 170. The data accumulated can then be sent to a user managed data base 172 or website or to a third party database 174 for storage and/or processing the data. Treatment protocols can be changed in accordance with the data.

Accordingly, an appliance has been disclosed which comprises a mask unit which covers a portion of the face of the user, leaving openings for the eyes. The mask is held in place by a stretchable band which fits around the head of the user, keeping the mask and the treatment elements in place so that the appliance can be hands free and permitting mobility of the user during treatment.

Although a preferred embodiment of the invention has been disclosed for purposes of illustration, it should be understood that various changes, modifications and substitutions may be incorporated in the embodiment without departing from the spirit of the invention, which is defined by the claims which follow.

What is claimed is:

1. A mask assembly for treatment of facial skin conditions, comprising:
a mask member which fits over a portion of the face of a user including skin surface around eyes of the user;
a retaining member for holding the mask member in place on a users' head;
eye portions allowing the user to see while wearing the mask member;
at least one treatment mechanism positioned on an interior surface of the mask for skin treatment, including treatment around the user's eyes; and
a mechanism for controlling the treatment mechanism.

2. The mask assembly of claim 1, wherein the treatment mechanism includes a plurality of LEDs and sealing elements around the eye portions to prevent LED light from reaching the eyes.

3. The mask assembly of claim 1, wherein the mask member is a 3D printed mask configured to fit an individual user's face.

4. The mask assembly of claim 3, including printed waveguides or light pipes on the mask member.

5. The mask assembly of claim 2, wherein the LEDs are individually controlled.

6. The mask assembly of claim 2, wherein the LEDs are positioned and controlled in groups.

7. The mask assembly claim 1, wherein the treatment mechanism is a plurality of message elements for massaging the skin and wherein the control system includes a motor for operating the message elements.

8. The mask assembly of claim 7, wherein the massage elements are mounted to oscillate.

9. The mask assembly of claim 1, elements wherein the treatment mechanism includes a plurality of vacuum elements positioned on the interior surface of the mask member.

10. The mask assembly of claim 1, including wherein the treatment mechanism including a plurality of microcurrent elements.

11. The mask assembly of claim 1, wherein the system for controlling includes a remote control member.

12. The mask assembly of claim 11, wherein the remote control unit is a smart phone with a Bluetooth or wifi communication with the control unit on the mask assembly.

13. The mask assembly of claim 12, including a plurality of treatment mechanism and the remote control unit includes the capability of selecting from among the plurality of treatments mechanisms.

14. The mask assembly of claim 1 , wherein the mask at least one data collection member for monitoring a selected skin condition.

15. The mask assembly of claim 14, including a storage member for storing information from the data collection member.

16. The mask assembly of claim 14, wherein the data collection member includes a camera and/or one or more skin condition sensors.

17. The mask assembly of claim 14, wherein the skin conditions include one or more of the following; hydration, wrinkles, oil production, color and elasticity.

18. The mask assembly of claim 1, including a battery and/or a connection to another source of power.

19. The mask assembly of claim 1, wherein the retaining member is a stretchable band, supporting the control system.

20. The mask assembly of claim 1, including a seal around the periphery of the mask.
